(19) United States
(12) Patent Application Publication
(10) Pub. No.: US 2025/0271377 A1
(43) Pub. Date: Aug. 28, 2025

(54) ACTIVATED CLOTTING TIME INSTRUMENT WITH ELECTRICAL IMPEDANCE TOMOGRAPHY DISPLAY

(71) Applicant: Medtronic, Inc., Minneapolis, MN (US)

(72) Inventors: Douglas J. Zimmerman, Fridley, MN (US); Charlene X. Yuan, Plymouth, MN (US)

(21) Appl. No.: 19/059,926

(22) Filed: Feb. 21, 2025

Related U.S. Application Data
(60) Provisional application No. 63/556,956, filed on Feb. 23, 2024.

(51) Int. Cl. G01N 27/02; G01N 33/86

(52) U.S. Cl. CPC G01N 27/02; G01N 33/86

(57) ABSTRACT

An analytical instrument configured to determine an ACT number of a blood sample based at least in part on a sequence of tomograms generated from electrical impedance tomography measurements performed in a test vessel on a fluid sample produced by mixing the blood sample and an activator of coagulation. In some examples, the ACT number is determined by applying image processing to the sequence of tomograms to determine the intensity of a blood clot in the fluid sample as a function of time. In some other examples, the ACT number is determined from time-dependent impedance profiles of the fluid sample measured via different pairs of electrodes of the test vessel, with the sequence of tomograms being used as an aid to interpretation of possible differences between different ones of the time-dependent impedance profiles.

### CROSS-REFERENCE TO RELATED APPLICATIONS

This application claims the benefit U.S. Provisional Patent Application No. 63/556,956, filed Feb. 23, 2024, and entitled “ACTIVATED CLOTTING TIME INSTRUMENT WITH ELECTRICAL IMPEDANCE TOMOGRAPHY DISPLAY,” the contents of which are incorporated herein by reference.

### FIELD

This application relates generally to activated clotting time instruments and methods.

### BACKGROUND

Activated clotting time (ACT) is used, for example, in cardiac surgery and interventional cardiology to assess unfractionated heparin activity. An ACT instrument derives an ACT number by exposing blood to an activator of coagulation (e.g., diatomaceous earth, kaolin, or glass particles) and measuring the time to form a fibrin clot. A representative ACT-number reference range is from 60 to 180 seconds. In a typical medical use case, ACT measurements are useful for preventing bleeding complications, which constitute a main adverse event of anticoagulant therapy and are important determinants of outcomes after the primary percutaneous coronary intervention (PCI) in acute myocardial infarction.

### SUMMARY

Disclosed herein are, among other things, various examples, aspects, features, and embodiments of an analytical instrument configured to determine an ACT number of a blood sample based at least in part on a sequence of tomograms generated from electrical impedance tomography (EIT) measurements performed in a test vessel on a fluid sample produced by mixing the blood sample and an activator of coagulation. In some examples, the ACT number is determined by applying image processing to the sequence of tomograms to determine the intensity of a blood clot in the fluid sample as a function of time. In some other examples, the ACT number is determined from time-dependent impedance profiles of the fluid sample measured via different pairs of electrodes of the test vessel, with the sequence of tomograms being used as an aid to interpretation of possible differences between different ones of the time-dependent impedance profiles.

According to one example, an analytic instrument includes a first test vessel having a plurality of electrodes arranged along a periphery thereof and configured to be in electrical contact with a fluid sample located in the first test vessel. The analytic instrument also includes an electrical measurement system configured to perform electrical impedance measurements on the fluid sample via the plurality of electrodes and a switching multiplexer configured to switch electrical connections between the electrical measurement system and different electrode pairs selected from the plurality of electrodes. The analytic instrument also includes an electronic controller configured to: (i) cause the electrical measurement system with the switching multiplexer to acquire sets of EIT data using the plurality of electrodes, with each of the sets corresponding to a different respective time, (ii) generate a plurality of tomograms of the fluid sample, with each of the tomograms being generated based on a corresponding one of the sets of EIT data, and (iii) arrange the plurality of tomograms into an ordered sequence based on the different respective times.

According to another example, a method of determining an ACT number corresponding to a blood sample includes mixing the blood sample the with an activator of coagulation in a test vessel having a plurality of electrodes arranged along a periphery thereof and configured to be in electrical contact with a resulting fluid sample. The method also includes, with an electrical measurement system, performing electrical impedance measurements on the fluid sample via the plurality of electrodes and, with a switching multiplexer, switching electrical connections between the electrical measurement system and different electrode pairs selected from the plurality of electrodes. The method also includes, with an electronic controller, (i) causing the electrical measurement system with the switching multiplexer to acquire sets of EIT data using the plurality of electrodes, with each of the sets corresponding to a different respective time, (ii) generating a plurality of tomograms of the mixed sample, with each of the tomograms being generated based on a corresponding one of the sets of EIT data, and (iii) arranging the plurality of tomograms into an ordered sequence based on the different respective times. The ACT number corresponding to the blood sample is determined using at least a portion of the ordered sequence.

### BRIEF DESCRIPTION OF THE DRAWINGS

FIG. 1 is a block diagram illustrating an ACT instrument according to various examples.

FIGS. 2A-2B are block diagrams illustrating a sample cartridge used in the ACT instrument of FIG. 1 according to various examples.

FIG. 3 is a flowchart of illustrating an ACT-measurement method implemented using the ACT instrument of FIG. 1 according to various examples.

FIG. 4 is a flowchart illustrating a data-acquisition block used in the ACT-measurement method of FIG. 3 according to some examples.

FIG. 5 shows representative frames of a clotting video generated using the method of FIG. 3 according to one example.

FIG. 6 is a flowchart illustrating an ACT/clot lysis time (CLT)-measurement method implemented using the ACT instrument of FIG. 1 according to various examples.

### DETAILED DESCRIPTION

In some examples, derivation of ACT values is based on electrical impedance measurements that use two or more electrodes in electrical contact a blood sample to obtain impedance data for the sample located in a test vessel. A disposable cartridge used for the measurements typically has several sample vessels. Different sample vessels may contain differing amounts of reagent which reacts with the blood sample deposited into the vessel. The measured impedance response of the sample vessel is fed into a suitable algorithm that processes time-resolved impedance data to determine the clotting time corresponding to that vessel. Without more, this approach provides ACT numbers but no additional insight into the clot formation process or the clot structure.

Electrical impedance tomography (EIT) is a method that uses more than two (for example, eight, sixteen, or more) electrodes arranged around the periphery of a sample vessel to measure electrical signals between different pairs of electrodes. The resulting sets of EIT data are processed through an inverse transform to construct a spatially resolved, e.g., two-dimensional (2D), cross-sectional map of the sample. This process can be periodically repeated for the sample vessel to obtain a time-resolved sequence of such cross-sectional maps that can be assembled into a video clip.

An example ACT instrument described herein below can be configured to perform both electrical-impedance and EIT measurements on the same set of sample-loaded test vessels. In some examples, the ACT instrument uses the acquired EIT data to create a clotting video visualizing clot growth in the sample vessel(s) over time for presentation to the user. In some examples, the clotting video can beneficially be used to provide an accelerated view of an estimate of the clotting time in real-time. In some other examples, the clotting video can beneficially be used to provide disambiguation of the ACT numbers obtained via electrical-impedance measurements when electrical-impedance data alone do not lend themselves to a clearcut interpretation for medical purposes. In yet some other examples, the ACT instrument operates to determine ACT numbers based on image processing applied to the clotting videos of different sample-loaded test vessels.

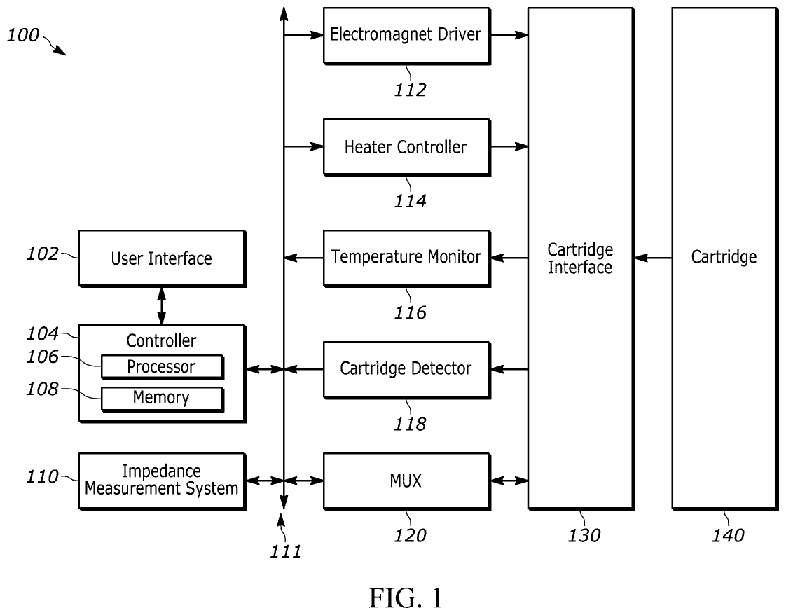

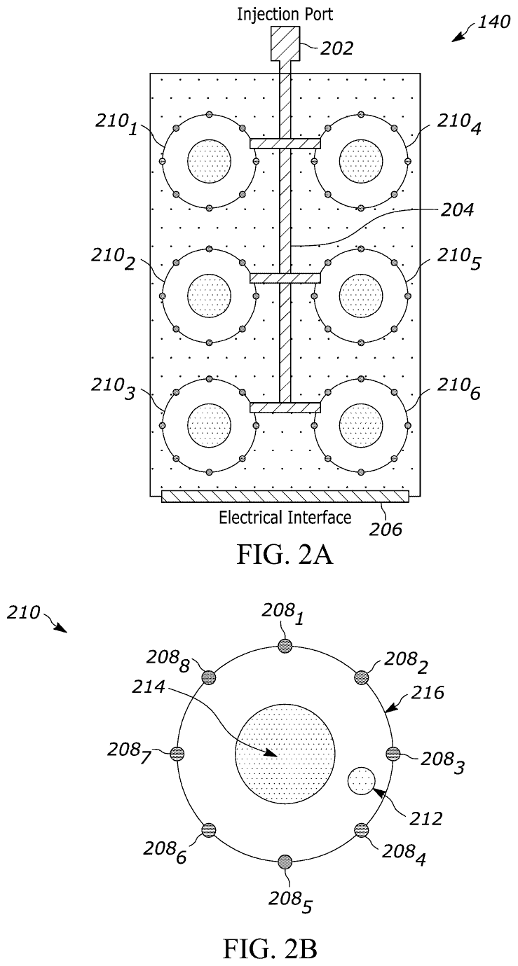

FIG. 1 is a block diagram illustrating an ACT instrument 100 according to some examples. The ACT instrument 100 includes a cartridge 140 having one or more sample vessels (note explicitly shown in FIG. 1, see, e.g., elements 210 in FIGS. 2A-2B). In a representative example, each of the sample vessels of the cartridge 140 is outfitted with a respective plurality of electrodes suitable for electrical-impedance and EIT measurements on the sample placed into the vessel. Various examples of the cartridge 140 and its constituent sample vessels are described in more detail below in reference to FIGS. 2A-2B.

The cartridge 140 is connected to a cartridge interface 130 configured to support electrical, mechanical, and fluid communications between the cartridge 140 and various other components of the ACT instrument 100. In some examples, the cartridge interface 130 includes a mechanical cartridge holder configured to accept and hold the cartridge 140 in place. In some examples, the mechanical cartridge holder incorporates one or more mechanical actuators or transducers that can vibrate the cartridge 140 as a whole or selected individual sample vessels of the cartridge 140. The cartridge interface 130 also includes electrical connectors for electrically connecting various electrodes of the sample vessels, via a multiplexer (MUX) 120, to an impedance measurement system 110 of the ACT instrument 100. In various examples, the MUX 120 includes a switch matrix that can be used to controllably switch various electrical connections between the impedance measurement system 110 and different electrode pairs of the sample vessels, e.g., as directed by instrument-control software during ACT and EIT measurements. In the example shown, the cartridge interface 130 is also connected to an electromagnetic driver 112, a heater controller 114, a temperature monitor 116, and a cartridge detector 118.

The electromagnetic driver 112 is configured to generate variable magnetic fields at the sample vessel(s) of the cartridge 140. In operation, a variable magnetic field generated by the electromagnetic driver 112 interacts with a magnetic target located in the corresponding sample vessel of the cartridge 140 to cause the magnetic target to move and stir the contents of the sample vessel. The heater controller 114 is configured to regulate electrical currents applied to one or more electrical heaters such that the sample vessels of the cartridge 140 are maintained at a selected fixed temperature. In various examples, the electrical heaters may be arranged in and around the mechanical cartridge holder, e.g., to form a thermal jacket, and/or incorporated into the cartridge 140. The temperature monitor 116 is configured to monitor the temperature of the cartridge 140. In some examples, such temperature monitoring is performed using one or more thermistors thermally coupled to the cartridge 140. The temperature readouts obtained with the temperature monitor 116 are used to provide feedback to the heater controller 114 such that the selected temperature can be accurately maintained. The cartridge detector 118 is configured to provide an electronic controller 104 of the ACT instrument 100 with an indication of whether the cartridge 140 is installed and properly connected to the cartridge interface 130. The impedance measurement system 110 is configured to measure electrical currents, voltages, and impedances corresponding to different selected pairs of electrodes of the cartridge 140 at various frequencies, e.g., as directed by the electronic controller 104. Example operations performed by the impedance measurement system 110 include but are not limited to:
(i) generating a dc bias voltage; (ii) generating an ac voltage or current having a selected frequency and amplitude; (iii) changing the frequency of the ac voltage or current; (iv) sensing an electrical current flowing between electrodes in response to the applied voltage; and (v) sensing a voltage between electrodes in response to the electrical current driven therethrough.

For impedance measurements, the impedance measurement system 110 is configured to apply an ac voltage to a selected pair of electrodes of the sample vessel or to drive an ac current to a selected pair of electrodes of the sample vessel. Then, using measurements of the corresponding current or voltage, the impedance measurement system 110 determines and records the complex electrical impedance (e.g., in the form of magnitude and phase angle) corresponding the selected pair of electrodes as a function of time. The electronic controller 104 uses an ACT-number determination algorithm to process and analyze the resulting recorded time-dependent impedance profile of the sample and to determine the corresponding ACT number based on such processing and analysis. In some examples, the ACT-number determination algorithm is configured to determine the ACT number based on a point in time at which the slope of the time-dependent impedance curve changes from a first value to a different second value in a substantially step-like manner. In some other examples, the ACT-number determination algorithm is configured to determine the ACT number using one or more other features of the correlation between blood clotting and time-dependent impedance changes, which are well established in the pertinent literature.

For EIT measurements, the impedance measurement system 110 is configured to apply an ac current to a selected pair of electrodes (sometimes referred to as the “driving electrode pair”) of the sample vessel and to measure and record the resulting electrical potentials (or voltages) induced at the other electrodes of that sample vessel. The electronic controller 104 then causes the impedance measurement system 110 and the MUX 120 to change the driving electrode pair by reconfiguring the switch matrix of the MUX 120. Thereafter, the impedance measurement system 110 applies an ac current to the new driving electrode pair and records the resulting electrical potentials from the other electrodes. This process is repeated numerous times by cycling through different driving electrode pairs. The electronic controller 104 then runs an image reconstruction algorithm to process the body of EIT data acquired in this manner and to compute therefrom a time series of 2D tomograms of the sample vessel. In some examples, the time series of 2D tomograms is presented to the user of the ACT instrument 100 in the form of the above-mentioned clotting video.

In the example shown, the electronic controller 104 is connected to the impedance measurement system 110, electromagnetic driver 112, heater controller 114, temperature monitor 116, cartridge detector 118, and MUX 120 via a communication interface (e.g., a system bus) 111 and includes a processor 106 and a memory 108. The communication interface 111 enables the electronic controller 104 to receive various input signals and to output various output signals from/to other components of the of the ACT instrument 100. The memory 108 has buffers to receive data representing the above-described impedance and EIT measurements. Once the data are received, the memory 108 may provide parts of the data to the processor 106 for processing therein. The memory 108 also stores therein program code, which when executed by the processor 106 enables the electronic controller 104 to perform various data processing and instrument control operations, including but not limited to various operations of the instrument control software and the above-mentioned ACT-number determination and image reconstruction algorithms.

The electronic controller 104 is also connected to a user interface 102. In some examples, the user interface 102 includes a display device (e.g., one or multiple individual display devices) on which the above-mentioned time-dependent impedance profiles (curves) and corresponding ACT numbers can be displayed, the above-mentioned clotting video can be played, and/or individual frames of the clotting video can be presented. In various examples, the display device may include any visual indicators, such as a heads-up display, a computer monitor, a projector, a touchscreen display, a liquid crystal display (LCD), a light-emitting diode display, or a flat panel display. The user interface 102 may also include one or more input/output (I/O) devices, such as one or more data/signal transfer interfaces, audio I/O devices (e.g., microphones or microphone arrays, speakers, headsets, earbuds, alarms, etc.), printers, sensors (e.g., temperature sensors, humidity sensors, pressure sensors, vibration sensors, etc.), image capture devices (e.g., one or more cameras), human interface devices (e.g., keyboards, cursor control devices, such as a mouse, a stylus, a trackball, or a touchpad), etc.

FIGS. 2A-2B are block diagrams illustrating the cartridge 140 according to some examples. More specifically, FIG. 2A shows a top (plan) view of the cartridge 140. FIG. 2B shows a top view of an individual sample well (vessel) 210 of the cartridge 140. In the example shown, the cartridge 140 has six sample wells 210, which are labeled in FIG. 2A with the reference numerals 210₁-210₆, respectively. In other examples, the cartridge 140 may have a different (from six) number of sample wells 210. In some examples, the cartridge 140 may have a single sample well 210. In some examples, the cartridge 140 is a disposable cartridge, which is intended for a single use.

The cartridge 140 has an injection port 202 for connecting an external fluid injector, such as a syringe, a pipette, or an automated injector device. The injection port 202 is connected to a capillary 204 configured to provide fluid pathways between the injection port 202 and each of the sample wells 210₁-210₆. In some examples, the shape and configuration of the capillary 204 is such that a blood volume transferred from the external fluid injector into the injection port 202 is distributed approximately equally between the sample wells 210₁-210₆.

In the example shown, a sample well 210 has a substantially cylindrical shape and, as such, has a circular cross-section in the top views shown in FIGS. 2A-2B. The sample well 210 has a plurality of electrodes 208₁-208_{N} arranged along a cylindrical sidewall 216 thereof to be in electrical contact with the sample. In the example shown, the number N is N=8. In other examples, the number N can be different from eight, e.g., greater or smaller than eight.

In some examples, an electrode 208ₙ includes a vertically oriented wire attached to the cylindrical sidewall 216 of the corresponding sample well 210. The sample well 210 also includes a ground electrode 212, which may be a wire of a different (e.g., larger) diameter than that of the wire electrodes 208₁-208_{N}. In some examples, the sample well 210 may have two or more ground electrodes 212. When the cartridge 140 is installed into the cartridge holder of the cartridge interface 130, the electrodes 208ₙ, 212 become electrically connected, via the cartridge's electrical interface 206, to the MUX 120. In some examples, the electrical interface 206 may incorporate full or partial MUX functionality to reduce the number of electrical connections between the impedance measurement system 110 and the cartridge interface 130. In some examples, any pair of the electrodes 208ₙ selected from the plurality of electrodes 208₁-208_{N} can serve as impedance-measurement electrodes (i.e., the electrodes used for the above-described time-resolved impedance measurements) or as driving electrodes (i.e., the electrodes used to drive the EIT measurements).

In some examples, the sample wells 210₁-210₆ contain pre-dispensed (e.g., dry) blood-activation reagents. Different ones of the sample wells 210₁-210₆ may contain different respective blood-activation reagents, different amounts of the same blood-reacting reagent, or reagents that interact with pharmaceuticals, such as an anticoagulant, in blood. When a blood sample is injected into the sample wells 210₁-210₆ through the capillary 204, the pre-dispensed blood-activation reagents are mixed in and react with the blood sample. In some examples, the mixing is facilitated using a respective movable magnetic target 214 located in each of the sample wells 210₁-210₆. In such examples, the magnetic target 214 is magnetically agitated or otherwise moved within the sample well 210 by a rotating or changing magnetic field generated with the electromagnetic driver 112, thereby stirring the blood sample and reagents located in the sample well 210. In a representative example, the magnetic target 214 is electrically insulated from the sample and the electrodes 208ₙ, 212 to prevent interference with the impedance and EIT measurements.

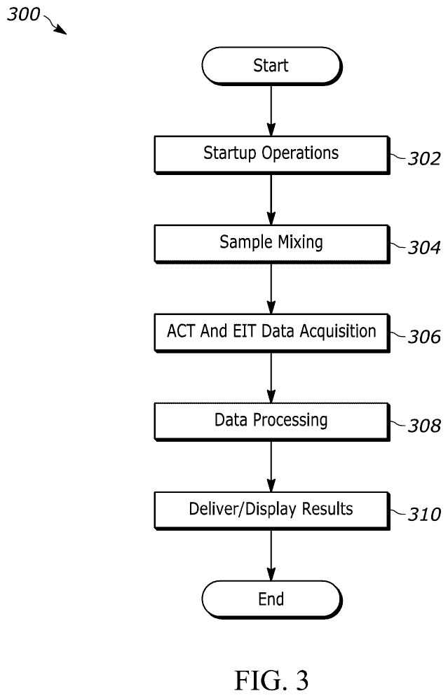

FIG. 3 is a flowchart illustrating an ACT-measurement method 300 implemented using the ACT instrument 100 according to various examples. The method 300 is described below in continued reference to FIGS. 1-3.

The method 300 includes a startup block 302. Example operations of the startup block 302 include one or more of the following operations. The user of the ACT instrument 100 turns on the ACT instrument 100 and waits for the various components of the instrument to become ready for measurements. For example, during such wait time, the electronic controller 104 boots up, and the heater controller 114 starts to regulate the temperature to bring the cartridge holder or thermal jacket of the cartridge interface 130 to a specified temperature, e.g., 37° C. The user acquires a blood sample and injects the acquired blood sample into the injection port 202 of the cartridge 140. The user then inserts the cartridge 140 into the cartridge holder of the cartridge interface 130 such that the cartridge detector 118 provides an indication of proper connection/coupling of the cartridge 140 to the relevant components of the ACT instrument 100. After the cartridge detector 118 signals that the cartridge 140 is properly installed into the cartridge interface 130, the electronic controller 104 prompts the user to initiate the measurements.

The method 300 also includes a mixing block 304. Operations of the mixing block 304 include the electronic controller 104 activating the electromagnetic driver 112. The activated electromagnetic driver 112 generates changing magnetic fields that interact with and agitate the magnetic targets 214 located in individual sample wells 210 of the cartridge 140. The agitated magnetic targets 214 stir the blood samples in the sample wells 210, thereby mixing the blood samples with the respective pre-dispensed blood-activation reagents located in those sample wells. The electronic controller 104 deactivates the electromagnetic driver 112 after a fixed amount of time. After the deactivation, the magnetic targets 214 stop moving and fall to the bottom of the corresponding sample wells 210.

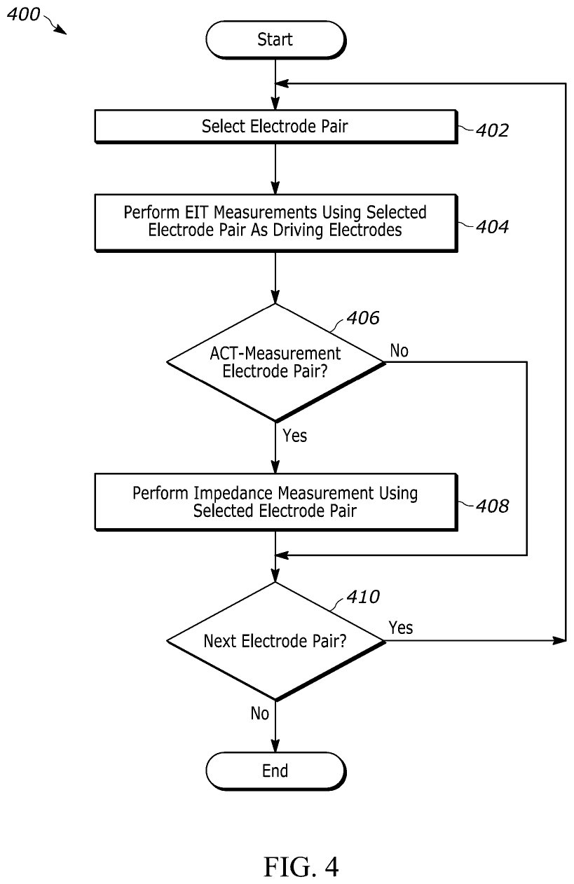

The method 300 also includes a data acquisition block 306. Operations of the data acquisition block 306 include the electronic controller 104 controlling the impedance measurement system 110 and the MUX 120 such that time-resolved impedance and EIT measurements are performed on each of the samples located in the different ones of the sample wells 210. More specifically, for each sample well 210, the impedance measurements are directed at recording one or more time-dependent impedance profiles (curves) of the sample, and the EIT measurements are directed at acquiring EIT data from which cross-sectional maps of the sample can be constructed and then used as frames of the corresponding clotting video. In a representative example, the impedance and EIT measurements in each of the sample wells 210 are time interleaved and include the MUX 120 going through a sequence of different impedance-measurement and driving electrode pairs selected from the corresponding plurality of electrodes 208₁-208_{N}. A non-limiting example of such sequence is described in more detail in reference to FIG. 4. Based on the provided description, a person of ordinary skill in the pertinent art will be able to make and use other suitable sequences without any undue experimentation.

The method 300 also includes a data processing block 308. Operations of the data processing block 308 include the electronic controller 104 processing the impedance and EIT data acquired during the data acquisition block 306 to construct, for each sample well 210, one or more respective time-dependent impedance profiles (curves) and a respective clotting video. For example, for a selected pair of impedance-measurement electrodes of the sample well 210, the electronic controller 104 constructs the corresponding time-dependent impedance profile by plotting the measured impedance values as a function of time in accordance with the timestamps of the individual impedance measurements performed using that pair of electrodes. The electronic controller 104 then analyzes the time-dependent impedance profile in accordance with the applicable ACT-number determination algorithm to determine therefrom the corresponding clotting time. To obtain a frame for the clotting video of the sample well 210, the electronic controller 104 applies an inverse transform function to the EIT data set corresponding to a sequence of different unique pairs of driving electrodes selected from the electrodes 208₁-208_{N} of that sample well 210. In some examples, electronic controller 104 applies image processing to obtain an ACT number from the clotting video.

The method 300 also includes a result delivery block 310. Operations of the result delivery block 310 include the electronic controller 104 displaying some or all of the clotting time results via the user interface 102. An option is provided to the user to review individual frames of the clotting videos or to playback the whole clotting videos corresponding to different sample wells 210 via the user interface 102. Upon completion of the operations of the result delivery block 310, the method 300 is terminated.

As already mentioned above, in at least some examples, any pair of electrodes from the set of electrodes 208₁-208_{N} of a sample well 210 can be used as an impedance-measurement electrode pair (i.e., a pair of electrodes used for measuring a time-dependent impedance profile of the sample). Similarly, any pair of electrodes from the set of electrodes 208₁-208_{N} of a sample well 210 can be used as driving electrodes (i.e., the electrodes used to drive EIT measurements). To acquire a set of EIT data sufficient for constructing a single cross-sectional map of the sample, the electronic controller 104 typically causes the MUX 120 to sequentially use all possible unique pairs (208ₘ, 208ₙ) of the electrodes 208₁-208_{N} of the same sample well 210 as driving electrodes. The number of such unique pairs is given by the binomial coefficient C_{N}². For example, for N=8, the number of unique electrode pairs is C₈²=28. As another example, for N=16, the number of unique electrode pairs is C₁₆²=120.

Unlike the EIT measurements, a time-dependent impedance profile is typically measured using a fixed pair of electrodes selected from the electrodes 208₁-208_{N} of the sample well 210. Better measurement results are typically produced when the two selected electrodes are opposite one another across the sample well 210 such that the electrical current flowing between the electrodes is substantially delocalized and has sizable components that flow through substantially all parts of the sample. An example of such pair of opposite electrodes for the sample well 210 illustrated in FIG. 2B is the electrode pair (208₁, 208₅) or the electrode pair (208₂, 208₆), or the like. For N=8, there are four different pairs of opposite electrodes. For N=16, there are eight different pairs of opposite electrodes. It should be noted that inconsistent ACT measurement results may be produced when the two selected electrodes are immediately adjacent with one another along the sidewall 216. An example of such pair of adjacent electrodes for the sample well 210 illustrated in FIG. 2B is the electrode pair (208₁, 208₂) or the electrode pair (208₂, 208₃), or the like. In various examples, some specific electrode pair (208ₘ, 208ₙ), e.g., a pair of opposite electrodes, may be selected and designated as an impedance-measurement electrode pair.

In some examples, to acquire multiple time-dependent impedance profiles in the same sample well 210, multiple pairs (208ₘ, 208ₙ) of opposite electrodes may be selected and designated as impedance-measurement electrode pairs, and the corresponding impedance measurements may be performed in the data acquisition block 306 via different impedance-measurement electrode pairs in a time-interleaved manner. Each impedance-measurement electrode pair will thus produce a separate respective time-dependent impedance profile at the end of the data acquisition block 306. In the data processing block 308, such time-dependent impedance profiles can be independently processed to determine respective ACT numbers. The ACT numbers corresponding to different ACT-measurement electrode pairs can then be compared and analyzed, e.g., to identify outliers and/or to spot possible inconsistencies. The corresponding clotting video may further be used to discard some of the ACT numbers and/or to select a subset of the ACT numbers for being delivered to the user in the result delivery block 310. In some examples, a weighted average of the ACT numbers obtained from different impedance-measurement electrode pairs can be computed and presented to the user in the result delivery block 310.

FIG. 4 is a flowchart illustrating a data-acquisition method 400 used in the data acquisition block 306 of the method 300 according to some examples. The method 400 is directed to acquiring both impedance and EIT data from a single sample well 210. The EIT data acquired with the method 400 are typically sufficient to construct a single frame of the corresponding clotting video. The method 400 can be repeated multiple times to acquire the EIT data for constructing a video sequence for the clotting video.

The method 400 includes the electronic controller 104 selecting a next electrode pair from the electrodes 208₁-208_{N} of the sample well 210 (in a block 402). In the first instance of the block 402, any electrode pair can be selected. In any subsequent instance of the block 402, any previously selected electrode pairs are removed from the list of electrode pairs available for selection, and a next electrode pair is selected from the electrode pairs remaining on the list.

The method 400 also includes the ACT instrument 100 performing EIT measurements (in a block 404). The EIT measurements of the block 404 are performed using the electrode pair selected in the block 402 as the driving electrode pair. The corresponding obtained EIT data are time stamped and saved in the memory 108 for processing in the data processing block 308 of the method 300.

The method 400 also includes the electronic controller 104 determining whether the electrode pair selected in the block 402 is designated as an impedance-measurement electrode pair (in a decision block 406). When the electronic controller 104 determines that the electrode pair selected in the block 402 is an impedance-measurement electrode pair (“Yes” at the decision block 406), the processing of the method 400 is directed to a block 408. When the electronic controller 104 determines that the electrode pair selected in the block 402 is not an impedance-measurement electrode pair (“No” at the decision block 406), the processing of the method 400 is directed to a decision block 410.

Operations of the block 408 include the ACT instrument 100 performing an impedance measurement using the electrode pair selected in the block 402. The corresponding obtained impedance data are time stamped and saved in the memory 108 for constructing a corresponding time-dependent impedance profile in the data processing block 308 of the method 300. In some examples, an impedance measurement is not performed in the block 408. Instead, the expected impedance data are extracted from the EIT data obtained for this electrode pair in the immediately preceding block 404.

Operations of the decision block 410 include the electronic controller 104 determining whether to select a next electrode pair. In some examples, this determination is made in the decision block 410 by determining whether the current list of electrode pairs available for selection is empty. When the electronic controller 104 determines that the selection list is not empty (“Yes” at the decision block 410), the processing of the method 400 is looped back to the block 402. When the electronic controller 104 determines that the selection list is empty (“No” at the decision block 410), the method 400 is terminated.

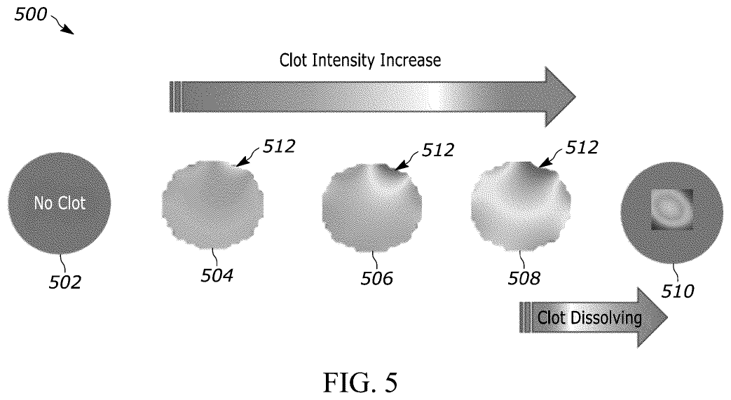

FIG. 5 shows several frames of a clotting video 500 generated with the ACT instrument 100 using the methods 300, 400 according to one example. More specifically, five individual video frames, labeled 502, 504, 506, 508, and 510, respectively, of the clotting video 500 are presented. The video frame 502 shows the 2D cross-sectional map of the sample at the beginning of the clotting process and indicates that the sample in the corresponding sample well 210 is homogeneous (has no clot). The video frames 504, 506, and 508 indicate a progressing clot growth, with a core 512 of the growing clot being located near the sidewall 216 of the corresponding sample well 210 at approximately 1-o'clock azimuthal angle. The video frame 510 indicates that the clot is dissolving due to the clot lysis.

The video frames 504, 506, and 508 clearly indicate an off-center clot location. Such location is likely to cause different pairs of opposite electrodes (208ₘ, 208ₙ), serving as different impedance-measurement electrode pairs, to produce different respective recorded time-dependent impedance profiles of the same sample. For example, for the indicated clot location, the electrode pair (208₂, 208₆) and the electrode pair (208₄, 208₈) are likely to detect markedly different time-dependent impedance profiles of the same sample, which are likely to result in different respective ACT numbers determined based on the processing and analysis of those impedance profiles in the data processing block 308 of the method 300. Without the clotting video 500, it might be difficult or impossible to disambiguate or properly interpret those ACT numbers. However, with the clotting video 500, the user can more easily interpret the observed differences in the determined ACT numbers. For example, based on the determined off-center location of the blood clot indicated by the frames 504-508 of the clotting video 500, the ACT numbers determined based on different time-dependent impedance profiles recorded with different impedance-measurement electrode pairs may be given different respective weights, and the final reported ACT number may be computed using the weighted average of those ACT numbers. In some cases, based on the clot geometry observed in the clotting video, some of those ACT numbers may be discarded (i.e., be given a zero weight).

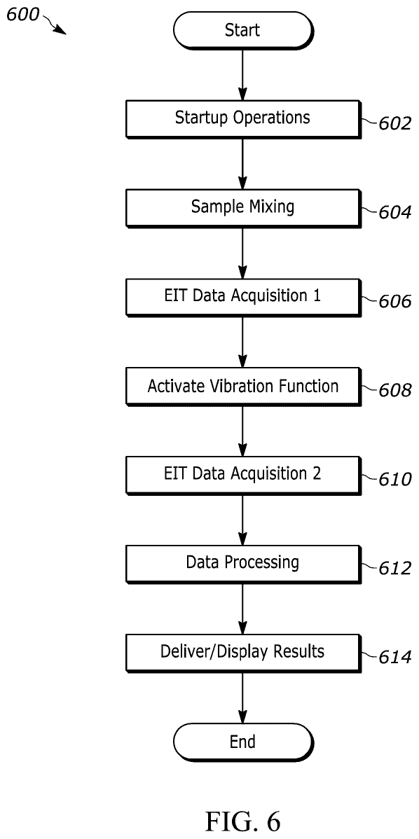

FIG. 6 is a flowchart illustrating an ACT/CLT-measurement method 600 implemented using the ACT instrument 100 according to various examples. Herein, the acronym CLT stands for “clot lysis time.” Some operations of the method 600 are substantially analogous to the corresponding operations of the method 300. The description of such operations is not repeated here, and the reader is referred to the corresponding parts of the description of the method 300 and FIG. 3. The description of the method 600 given below primarily focuses on differences between the methods 600 and 300. The method 600 is described below in continued reference to FIGS. 1-6.

A startup block 602 and a mixing block 604 of the method 600 are substantially analogous to the startup block 302 and the mixing block 304, respectively, of the method 300. In some examples, a data acquisition block 606 of the method 600 is substantially analogous to the data acquisition block 306 of the method 300. In some other examples, the data acquisition block 606 includes only the EIT measurements corresponding to the block 404 of the method 400 but does not include impedance measurements corresponding to the block 408 of the method 400.

The method 600 also includes the electronic controller 104 activating an ultrasound vibration function of the cartridge interface 130. When the ultrasound vibration function is activated, one or more ultrasonic actuators or transducers of the cartridge interface 130 vibrate the cartridge 140 as a whole or selected individual sample vessels of the cartridge 140 individually. As known in the pertinent art, ultrasound, either alone or enhanced by microbubbles and/or thrombolytic agents, can dissolve blood clots. In some examples, ultrasound energy causes vibrations that can either break the clot apart directly, e.g., via disruption of the fibrin matrix, or make the clot more susceptible to the effects of thrombolytic agents.

In some examples, a data acquisition block 610 of the method 600 is substantially analogous to the data acquisition block 306 of the method 300. In some other examples, the data acquisition block 610 includes only the EIT measurements corresponding to the block 404 of the method 400 but does not include impedance measurements corresponding to the block 408 of the method 400.

The method 600 also includes the electronic controller 104 carrying out data processing (in a block 612) of the data acquired in the data acquisition blocks 606, 610. Operations of the block 612 include the electronic controller 104 generating a clotting video by processing sets of the EIT data acquired in the data acquisition blocks 606, 610, e.g., as described above. In some examples, the clotting video generated in the block 612 may be qualitatively similar to the clotting video 500 (FIG. 5) and contain first and second frame sequences. The first frame sequence represents clot formation and may be similar to the frame sequence illustrated by the frames 504, 506, and 508 of the clotting video 500. The second frame sequence, which follows in time the first frame sequence, represents clot lysis and may contain frames that are qualitatively similar to the frame 510 of the clotting video 500 but with varying degrees of clot dissolution.

Operations of the data processing block 612 also include the electronic controller 104 applying image processing to the first sequence to determine the corresponding ACT number. In some examples, to determine the ACT number from the first sequence, the electronic controller 104 is programmed to: (i) compute estimates of the intensity, volume, or mass of the observed blood clot for different frames of the first sequence; (ii) plot the computed estimates of the intensity, volume, or mass as a function of time using the timestamps of the frames; and (iii) determine a relative time at which the intensity, volume, or mass of the observed blood clot crosses an applicable threshold value. The threshold value is an algorithm parameter that is determined through calibration and computer simulation. The relative time at which the threshold value is reached is deemed to be the measured ACT number.

Operations of the data processing block 612 also include the electronic controller 104 applying image processing to the second sequence to determine the corresponding CLT number. In some examples, to determine the CLT number from the second sequence, the electronic controller 104 is programmed to: (i) compute estimates of the intensity, volume, or mass of the observed blood clot for different frames of the second sequence; (ii) compute estimates of the position change of the core 512 of the blood clot relative to the position of the core 512 in the first frame of the second sequence; (iii) plot the computed estimates of the intensity, volume, or mass and of the position change as a function of time using the timestamps of the frames; (iv) determine a relative time at which the intensity, volume, mass, or position change reaches a respective applicable threshold value. The respective threshold values are algorithm parameters that are determined through calibration and computer simulation. The earliest relative time at which the respective threshold value is reached is deemed to be the measured CLT number.

The method 600 also includes a result delivery block 614. Operations of the result delivery block 614 include the electronic controller 104 displaying the measured ACT and CLT numbers via the user interface 102. An option is provided to the user to review individual frames of the clotting videos or to playback the whole clotting videos corresponding to different sample wells 210 via the user interface 102. Upon completion of the operations of the result delivery block 614, the method 600 is terminated.

According to an example embodiment disclosed above, e.g., in the summary section and/or in reference to any one or any combination of some or all of FIGS. 1-6, provided is an analytic instrument comprising: a first test vessel having a plurality of electrodes arranged along a periphery thereof and configured to be in electrical contact with a fluid sample located in the first test vessel; an electrical measurement system configured to perform electrical impedance measurements on the fluid sample via the plurality of electrodes; a switching multiplexer configured to switch electrical connections between the electrical measurement system and different electrode pairs selected from the plurality of electrodes; and an electronic controller configured to: cause the electrical measurement system with the switching multiplexer to acquire sets of EIT data using the plurality of electrodes, with each of the sets corresponding to a different respective time; generate a plurality of tomograms of the fluid sample, with each of the tomograms being generated based on a corresponding one of the sets of EIT data; and arrange the plurality of tomograms into an ordered sequence based on the different respective times.

In some embodiments of the above analytic instrument, the fluid sample includes a blood sample mixed with an activator of coagulation; and wherein the electronic controller is configured to determine an ACT number corresponding to the fluid sample using at least a first portion of the ordered sequence.

In some embodiments of any of the above analytic instruments, the analytic instrument is configured to subject the first test vessel to ultrasonic vibration; and wherein the electronic controller is configured to determine a CLT number corresponding to the fluid sample using a second portion of the ordered sequence, the second portion corresponding to a time interval of the ultrasonic vibration.

In some embodiments of any of the above analytic instruments, the electronic controller is configured to: apply image processing to the ordered sequence to determine one or more of intensity, volume, mass, and position change of a blood clot in the fluid sample as a respective function of time; and determine at least one of the ACT number and the CLT number by applying thresholding to one or more of the respective functions of time.

In some embodiments of any of the above analytic instruments, the electronic controller is further configured to cause the electrical measurement system with the switching multiplexer to record one or more time-dependent impedance profiles of the fluid sample, each of the one or more time-dependent impedance profiles being recorded using a different respective designated pair of electrodes selected from the plurality of electrodes.

In some embodiments of any of the above analytic instruments, the plurality of electrodes includes eight or more electrodes; and wherein a first designated pair of electrodes includes first and second electrodes of the plurality of electrodes that are opposite one another across a middle part of the first test vessel.

In some embodiments of any of the above analytic instruments, a second designated pair of electrodes includes third and fourth electrodes of the plurality of electrodes that are opposite one another across the middle part of the first test vessel.

In some embodiments of any of the above analytic instruments, the fluid sample includes a blood sample mixed with an activator of coagulation; and wherein the electronic controller is configured to determine an ACT number corresponding to the fluid sample using the one or more time-dependent impedance profiles.

In some embodiments of any of the above analytic instruments, the ordered sequence is used as an aid to interpretation of differences between different ones of the time-dependent impedance profiles.

In some embodiments of any of the above analytic instruments, the electronic controller is configured to compute the ACT number corresponding to the fluid sample as a weighted sum of respective ACT numbers determined from individual ones of the time-dependent impedance profiles, with weights for the weighted sum being determined based on one or more tomograms of the ordered sequence.

In some embodiments of any of the above analytic instruments, the electronic controller is configured to play the sequence on a user interface device as a video clip.

In some embodiments of any of the above analytic instruments, the first test vessel is one of a plurality of test vessels included in a cartridge.

In some embodiments of any of the above analytic instruments, the cartridge further includes: an injection port for connecting a fluid injector; and a capillary connecting the injection port to each test vessel of the plurality of test vessels.

In some embodiments of any of the above analytic instruments, the cartridge further includes an electrical interface configured to electrically connect the electrodes of different test vessels of the plurality of test vessels to the switching multiplexer.

In some embodiments of any of the above analytic instruments, the analytic instrument further comprises an electromagnetic driver, wherein each test vessel of the plurality of test vessels contains a respective magnetic target that is movable within the test vessel in response to a changing magnetic field generated with the electromagnetic driver.

In some embodiments of any of the above analytic instruments, the analytic instrument further comprises a temperature regulator configured to maintain a temperature of the cartridge at a selected fixed temperature value.

In some embodiments of any of the above analytic instruments, each test vessel of the plurality of test vessels contains a respective pre-dispensed reagent.

In some embodiments of any of the above analytic instruments, the respective pre-dispensed reagents of different test vessels have different respective amounts of a same chemical compound.

In some embodiments of any of the above analytic instruments, at least two of the respective pre-dispensed reagents differ in chemical composition.

According to another example embodiment disclosed above, e.g., in the summary section and/or in reference to any one or any combination of some or all of FIGS. 1-6, provided is a method of determining an ACT number of a blood sample, the method comprising: mixing the blood sample the with an activator of coagulation in a test vessel having a plurality of electrodes arranged along a periphery thereof and configured to be in electrical contact with a resulting fluid sample; with an electrical measurement system, performing electrical impedance measurements on the fluid sample via the plurality of electrodes; with a switching multiplexer, switching electrical connections between the electrical measurement system and different electrode pairs selected from the plurality of electrodes; and with an electronic controller, causing the electrical measurement system with the switching multiplexer to acquire sets of EIT data using the plurality of electrodes, with each of the sets corresponding to a different respective time; generating a plurality of tomograms of the mixed sample, with each of the tomograms being generated based on a corresponding one of the sets of EIT data; and arranging the plurality of tomograms into an ordered sequence based on the different respective times, wherein the ACT number corresponding to the blood sample is determined using at least a portion of the ordered sequence.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain implementations and should in no way be construed to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

Unless explicitly stated otherwise, each numerical value and range should be interpreted as being approximate as if the word “about” or “approximately” preceded the value or range.

The use of figure numbers and/or figure reference labels (if any) in the claims is intended to identify one or more possible embodiments of the claimed subject matter in order to facilitate the interpretation of the claims. Such use is not to be construed as necessarily limiting the scope of those claims to the embodiments shown in the corresponding figures.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a,” “has . . . a,” “includes . . . a,” “contains . . . a” does not, without more constraints, preclude the existence of additional identical elements in the process, method, article, or apparatus that comprises, has, includes, contains the element. Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

Unless otherwise specified herein, in addition to its plain meaning, the conjunction “if” may also or alternatively be construed to mean “when” or “upon” or “in response to determining” or “in response to detecting,” which construal may depend on the corresponding specific context. For example, the phrase “if it is determined” or “if [a stated condition] is detected” may be construed to mean “upon determining” or “in response to determining” or “upon detecting [the stated condition or event]” or “in response to detecting [the stated condition or event].”

Throughout the detailed description, the drawings, which are not to scale, are illustrative only and are used in order to explain, rather than limit the disclosure. The use of terms such as height, length, width, top, bottom, is strictly to facilitate the description of the embodiments and is not intended to limit the embodiments to a specific orientation. For example, height does not imply only a vertical rise limitation, but is used to identify one of the three dimensions of a three-dimensional structure as shown in the figures. Such “height” would be vertical where the electrodes are horizontal but would be horizontal where the electrodes are vertical, and so on. Similarly, while all figures show the different layers as horizontal layers such orientation is for descriptive purpose only and not to be construed as a limitation.

Also, for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. The same type of distinction applies to the use of terms “attached” and “directly attached,” as applied to a description of a physical structure. For example, a relatively thin layer of adhesive or other suitable binder can be used to implement such “direct attachment” of the two corresponding components in such physical structure.

The described embodiments are to be considered in all respects as only illustrative and not restrictive. In particular, the scope of the disclosure is indicated by the appended claims rather than by the description and figures herein. All changes that come within the meaning and range of equivalency of the claims are to be embraced within their scope.

The functions of the various elements shown in the figures, including any functional blocks labeled as “processors” and/or “controllers,” may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and nonvolatile storage. Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

As used in this application, the term “circuitry” may refer to one or more or all of the following: (a) hardware-only circuit implementations (such as implementations in only analog and/or digital circuitry); (b) combinations of hardware circuits and software, such as (as applicable): (i) a combination of analog and/or digital hardware circuit(s) with software/firmware and (ii) any portions of hardware processor(s) with software (including digital signal processor(s)), software, and memory(ies) that work together to cause an apparatus, such as a mobile phone or server, to perform various functions); and (c) hardware circuit(s) and or processor(s), such as a microprocessor(s) or a portion of a microprocessor(s), that requires software (e.g., firmware) for operation, but the software may not be present when it is not needed for operation.” This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in server, a cellular network device, or other computing or network device.

It should be appreciated by those of ordinary skill in the art that any block diagrams herein represent conceptual views of illustrative circuitry embodying the principles of the disclosure. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

What is claimed is:

1. An analytic instrument, comprising:
a first test vessel having a plurality of electrodes arranged along a periphery thereof and configured to be in electrical contact with a fluid sample located therein;
an electrical measurement system configured to perform electrical impedance measurements on the fluid sample via the plurality of electrodes;
a switching multiplexer configured to switch electrical connections between the electrical measurement system and different electrode pairs selected from the plurality of electrodes; and
an electronic controller configured to:
cause the electrical measurement system with the switching multiplexer to acquire sets of electrical impedance tomography (EIT) data using the plurality of electrodes, with each of the sets corresponding to a different respective time;
generate a plurality of tomograms of the fluid sample, with each of the tomograms being generated based on a corresponding one of the sets of EIT data; and
arrange the plurality of tomograms into an ordered sequence based on the different respective times.

2. The analytic instrument of claim 1,
wherein the fluid sample includes a blood sample mixed with an activator of coagulation; and
wherein the electronic controller is configured to determine an activated clotting time (ACT) number corresponding to the fluid sample using at least a first portion of the ordered sequence.

3. The analytic instrument of claim 2,
wherein the analytic instrument is configured to subject the first test vessel to ultrasonic vibration; and
wherein the electronic controller is configured to determine a clot lysis time (CLT) number corresponding to the fluid sample using a second portion of the ordered sequence, the second portion corresponding to a time interval of the ultrasonic vibration.

4. The analytic instrument of claim 3, wherein the electronic controller is configured to:
apply image processing to the ordered sequence to determine a parameter selected from the group consisting of intensity, volume, mass, and position change of a blood clot in the fluid sample as a function of time; and
determine at least one of the ACT number and the CLT number by applying thresholding to the function of time.

5. The analytic instrument of claim 1, wherein the electronic controller is further configured to cause the electrical measurement system with the switching multiplexer to record a first time-dependent impedance profile of the fluid sample using a first designated pair of electrodes selected from the plurality of electrodes.

6. The analytic instrument of claim 5,
wherein the plurality of electrodes includes eight or more electrodes; and
wherein the first designated pair of electrodes includes first and second electrodes of the plurality of electrodes that are opposite one another across a middle part of the first test vessel.

7. The analytic instrument of claim 6,
wherein the electronic controller is further configured to cause the electrical measurement system with the switching multiplexer to record a second time-dependent impedance profile of the fluid sample using a second designated pair of electrodes selected from the plurality of electrodes; and
wherein the second designated pair of electrodes includes third and fourth electrodes of the plurality of electrodes that are opposite one another across the middle part of the first test vessel.

8. The analytic instrument of claim 5,
wherein the fluid sample includes a blood sample mixed with an activator of coagulation; and
wherein the electronic controller is configured to determine an activated clotting time (ACT) number corresponding to the fluid sample using the first and second time-dependent impedance profiles.

9. The analytic instrument of claim 8, wherein the ordered sequence is used as an aid to interpretation of differences between different the first and second time-dependent impedance profiles.

10. The analytic instrument of claim 8, wherein the electronic controller is configured to compute the ACT number corresponding to the fluid sample as a weighted sum of respective ACT numbers determined from individual ones of the time-dependent impedance profiles, with weights for the weighted sum being determined based on one or more tomograms of the ordered sequence.

11. The analytic instrument of claim 1, wherein the electronic controller is configured to play the sequence on a user interface device as a video clip.

12. The analytic instrument of claim 1, wherein the first test vessel is one of a plurality of test vessels included in a cartridge.

13. The analytic instrument of claim 12, wherein the cartridge further includes:
an injection port for connecting a fluid injector; and
a capillary connecting the injection port to each test vessel of the plurality of test vessels.

14. The analytic instrument of claim 13, wherein the cartridge further includes an electrical interface configured to electrically connect the electrodes of different test vessels of the plurality of test vessels to the switching multiplexer.

15. The analytic instrument of claim 12, further comprising an electromagnetic driver,
wherein each test vessel of the plurality of test vessels contains a respective magnetic target that is movable within the test vessel in response to a changing magnetic field generated with the electromagnetic driver.

16. The analytic instrument of claim 12, further comprising a temperature regulator configured to maintain a temperature of the cartridge at a selected fixed temperature value.

17. The analytic instrument of claim 12, wherein each test vessel of the plurality of test vessels contains a respective pre-dispensed reagent.

18. The analytic instrument of claim 12, wherein the respective pre-dispensed reagents of different test vessels have different respective amounts of a same chemical compound.

19. The analytic instrument of claim 12, wherein at least two of the respective pre-dispensed reagents differ in chemical composition.

20. A method of determining an activated clotting time (ACT) number corresponding to a blood sample, the method comprising:
mixing the blood sample the with an activator of coagulation in a test vessel having a plurality of electrodes arranged along a periphery thereof and configured to be in electrical contact with a resulting fluid sample;
with an electrical measurement system, performing electrical impedance measurements on the fluid sample via the plurality of electrodes;
with a switching multiplexer, switching electrical connections between the electrical measurement system and different electrode pairs selected from the plurality of electrodes; and
with an electronic controller,
causing the electrical measurement system with the switching multiplexer to acquire sets of electrical impedance tomography (EIT) data using the plurality of electrodes, with each of the sets corresponding to a different respective time;
generating a plurality of tomograms of the mixed sample, with each of the tomograms being generated based on a corresponding one of the sets of EIT data; and
arranging the plurality of tomograms into an ordered sequence based on the different respective times,
wherein the ACT number corresponding to the blood sample is determined using at least a portion of the ordered sequence.
